(19) United States
(12) Patent Application Publication
(10) Pub. No.: US 2012/0191102 A1
(43) Pub. Date: Jul. 26, 2012

(54) MEDICINE INJECTION DEVICE AND METHOD

(75) Inventors: Atsushi MATSUMOTO, Tokyo (JP); Makoto Saruhashi, Tokyo (JP)

(73) Assignee: Terumo Kabushiki Kaisha, Shibuya-ku (JP)

(21) Appl. No.: 13/433,534

(22) Filed: Mar. 29, 2012

Related U.S. Application Data
(63) Continuation of application No. PCT/JP2010/066795, filed on Sep. 28, 2010.

(30) Foreign Application Priority Data
Sep. 29, 2009 (JP) 2009-224687

(51) Int. Cl. A61B 17/58

(52) U.S. Cl. 606/94

(57) ABSTRACT

A medicine injection device includes a medicine ejection unit having a fluid flow-in port for receiving liquid, and a pressure generation unit which includes a pressure generating chamber to generate pressure acting upon a working chamber of the medicine ejection unit through the liquid and which ejects medicine by moving a gasket based on the applied pressure. The pressure generation unit includes a pressure portion cylinder in the pressure generating chamber, a first pusher provided with a hollow gasket defining the inner volume of the pressure generating chamber and a second pusher which generates the pressure inside the pressure generating chamber by pressing a distal end head portion into the inside of the pressure generating chamber. The pressure portion cylinder and the first pusher are coupled through an engagement mechanism in which fixation and fixation release are possible at different pitches in the axial direction.

This application is a continuation of International Application No. PCT/JP2010/066795 filed on Sep. 28, 2010, and claims priority to Japanese Application No. 2009-224687 filed on Sep. 29, 2009, the entire content of both of which is incorporated herein by reference.

### TECHNICAL FIELD

The present invention relates to a medicine injection device for ejecting a medicine from a medicine ejection unit by pressure generated in a pressure generation unit and for injecting such medicine into a space.

### BACKGROUND DISCUSSION

In recent years, there has been carried out, with respect to compression fracture of a vertebral body caused by osteoporosis or cancer, percutaneous vertebroplasty (PVP) involving injecting a filling member which hardens over time after a bone biopsy needle is stuck into a bone fractured vertebral body. For this filling member, for example, there has been used calcium phosphate based bone cement, polymethyl methacrylate bone cement or the like having an X-ray contrast property (hereinafter, simply referred to also as “bone cement”). Usually, the bone cement has a very high viscosity and also, the inside of the vertebra which is filled with the bone cement has a very large pressure drop caused by a spongy substance or the like, so that during injection, it is necessary to inject the cement little by little with a high pressure of 3 MPa or more.

Therefore, for such an injection device, a constitution is used in which the bone cement is ejected while being applied with a high pressure by a general piston type or feed screw type syringe. On the other hand, a constitution has been proposed in which the bone cement is injected while suppressing the pressure to a certain degree by lowering the amount of injection gradually (see Japanese unexamined PCT patent publication No. 2004-507312).

As described above, for example, with respect to the medicine injection device used for injecting a liquid medicine (bone cement) having high viscosity into a space which has a high pressure drop such as the inside of the bone, it is desirable to employ a constitution in which the medicine can be ejected with high pressure by a proper amount every time.

However, it is difficult for an injection device of a usual piston type to generate an adequate high pressure and to inject medicine by a proper amount for every time depending on the generated high pressure. Also, it becomes possible for a feed screw type injection device to employ quantitative injection at a high pressure, but the high pressure state inside the cylinder is not released and is always maintained during the injection, so that in case of desiring to stop the injecting operation, it is difficult to stop it immediately. Further, in a constitution of injecting bone cement while suppressing the pressure to a certain degree by decreasing the amount of injection gradually such as a constitution described in Japanese unexamined PCT patent publication No. 2004-507312, there is a possibility that burden to a patient increases caused by the fact that the necessary time period until the injection completion lengthens and in addition, there is also a possibility that it is not possible to inject the necessary amount completely caused by the low pressure injection depending on the condition of the vertebral body or the like.

### SUMMARY

The medicine injection device disclosed here make it possible to inject or eject the medicine with a predetermined high pressure by a proper amount every time when injecting the medicine into the inside of a space, yet the pressure inside the cylinder is relatively easily releasable and the injection operation becomes easy-to-use.

A medicine injection device includes a medicine ejection unit in combination with a pressure generation unit. The medicine ejection unit comprises: a tubular body possessing an interior partitioned by a sub-gasket slidably arranged in a liquid-tight manner in the interior of the tubular body into a working chamber at a proximal portion of the tubular body and an action chamber positioned distally of the working chamber, and a fluid flow-in port in fluid communication with the working chamber. The pressure generation unit comprises: a pressure generating chamber which generates fluid pressure communicatable with the working chamber to act upon the working chamber and advance the sub gasket in a distal direction away from the fluid flow-in port; a pressure portion cylinder inside the pressure generating chamber; a first pusher which includes a main gasket slidably disposed in a liquid-tight manner in the pressure portion cylinder and defining an inner volume of the pressure generating chamber, the main gasket possessing a slide hole extending throughout the main gasket; and a second pusher passing through the first pusher and concurrently slidably positioned in a liquid-tight manner in the slide hole of the main gasket to generate the fluid pressure inside the pressure generating chamber by pressing a distal end head portion into the pressure generating chamber; and the pressure portion cylinder and the first pusher are coupled through an engagement mechanism configured to effect fixation between the pressure portion cylinder and the first pusher at a predetermined pitch in an axial direction and to effect fixation release between the pressure portion cylinder and the first pusher permitting relative movement between the pressure portion cylinder and the first pusher in the axial direction.

According to this construction, the sliding of the slide hole of the main gasket and the pressing the second pusher into the slide hole in a state in which both the pressure portion cylinder and the first pusher are fixed in the axial direction, it is possible to generate a relatively high pressure through the fluid in the pressure generating chamber and it is possible to make the sub gasket move by the generated pressure. Thus, it is possible to eject the medicine with a relatively high pressure from the medicine ejection unit in a proper amount every time and to inject the medicine into the inside of the non-injection space. Also, after the ejection, by making the main gasket progress or move inside the pressure generating chamber by shortening the space between the pressure portion cylinder and the first pusher through the engagement mechanism, it is possible for the second pusher to return to the original position and the pressure inside the system is reliably released simultaneously with the completion of the preparation of the next ejection process. Therefore, it is possible to prevent the pressure, which is generated inside the medicine ejection unit or inside the pressure generation unit by the pressing-in of the second pusher, from acting on a steady basis and it is possible to prevent erroneous medicine ejection on an occasion such as operation standby or the like.

When the region from the working chamber to the pressure generating chamber is filled with an incompressible fluid which is substantially not compressed by the pressure generated in the pressure generation unit, it is possible to drive the sub gasket without substantially losing the pressure generated in the pressure generation unit.

In this case, when the pressure portion cylinder communicates with the working chamber at the distal side thereof and concurrently, includes, at the proximal side thereof, a tubular portion formed with an opening end through which the main gasket is inserted; and the first pusher includes a first tubular portion on which the main gasket is fixed at the distal side thereof and which is insertable into the tubular portion, and a second tubular portion forming a tubular space into which the tubular portion is insertable with respect to the first tubular portion by being provided coaxially by sandwiching a predetermined space in the outer diameter direction of the first tubular portion, it is possible for the pressure portion cylinder to be inserted into the inside of the first pusher and to be coupled therewith relatively easily.

The engagement mechanism preferably includes a first rib which is provided on the outer circumferential surface of the tubular portion of the pressure portion cylinder and which projects from a portion in the circumferential direction of the outer circumferential surface toward the outer diameter direction, and plural second ribs having a predetermined pitch in the axial direction of the second tubular portion on an inner circumferential surface of the second tubular portion of the first pusher and which are engageable with the first rib for every pitch of the predetermined pitch in the axial direction of the second tubular portion by projecting from the portion in the circumferential direction of the inner circumferential surface toward the tubular space. The tubular space includes a rib path on the side of the second rib and in which the first rib is passable toward the axial direction of the second tubular portion. It is thus possible to realize the fixation and fixation release in the axial direction between the pressure portion cylinder and the first pusher by a simple constitution.

The first rib can be a plate-shaped member which is diameter-expanded for a portion of the outer circumferential surface of the tubular portion toward the outer diameter direction thereof and concurrently, is provided by at least one pair facing each other in the diameter direction of the tubular portion. The second rib is a plate-shaped member which is diameter-reduced for a portion of the inner circumferential surface of the second tubular portion toward the inner diameter direction thereof and concurrently, is provided by at least one pair facing to each other in the diameter direction of the second tubular portion. The rib path can be formed between the facing second ribs. It is thus possible to engage the first rib and the second rib at two places facing each other in the circumferential direction, so that it is possible to fix the pressure portion cylinder and the first pusher further tightly compared with a case, for example, in which each rib is installed by only one piece in the circumferential direction, and also endurance on an occasion of the pressure generation is improved.

It is possible to employ a configuration in which there is provided, for the second rib, a rib set by one pair facing to each other in the diameter direction of the second tubular portion in which a first small rib and a second small rib which are formed to be shorter than the circumferential direction length of the first rib are aligned in the circumferential direction of the inner circumferential surface of the second tubular portion. The first small ribs alone and the second small ribs alone can be respectively arranged to face each other between the facing rib sets, and in the axial direction of the second tubular portion, the pitch between the first small ribs alone and the pitch between the second small ribs alone are respectively set to be the predetermined pitches with which the first rib is engageable, and the pitches between the first small ribs and the second small ribs are set to be half the predetermined pitches. Thus, by rotating the pressure portion cylinder clockwise and counterclockwise alternately with respect to the first pusher, it is possible, while the first rib engages with respect to the first small rib and second small rib alternately, to relatively easily fix and move the pressure portion cylinder and the first pusher by an amount of the half pitch.

Both end portions of the first rib in the circumferential direction of the tubular portion can be provided with a first inclination surface whose width narrows toward the proximal side in the axial direction of the tubular portion. The end portion facing the rib path of the first small rib and the second small rib which constitute the rib set is configured as a second inclination surface whose width narrows toward the distal side in the axial direction of the second tubular portion and which the first inclination surface can move over by sliding. It is thus possible to carry out the fixation and movement of the pressure portion cylinder and first pusher by the half pitches further smoothly.

The first rib can be plural in number in the axial direction of the second tubular portion, having a predetermined pitch. The respective first ribs are engageable with the respective second ribs simultaneously so that the engagement strength, the stability on an occasion of the engagement or the like between the pressure portion cylinder and the first pusher is improved.

It is possible to employ a construction in which there is included a medicine ejection port in the action chamber of the tube body and the medicine is ejected from the medicine ejection port.

Also, if there is included a medicine container connection portion at the distal end portion of the tube body, and the sub gasket includes a plunger enterable into the inside of a medicine container which is connected to the action chamber side, it is possible to replace the medicine container easily and it is possible to improve the versatility and the usability of the medicine injection device furthermore.

There can also be included a flexible tube which provides communication between the working chamber of the medicine ejection unit and the pressure generating chamber of the pressure generation unit, and with the insides of the working chamber, the pressure generating chamber and the flexible tube filled with the incompressible fluid. It thus becomes possible, for example, in case of carrying out medicine injection into the inside of a bone under X-ray illumination or the like, to carry out injection operation by installing the pressure generation unit at a position apart from the medicine ejection unit which is installed near an X-ray tube.

The medicine injection device can be preferably used also in a case in which the medicine is bone cement to be injected into the inside of the bone. This is because it is necessary for the bone cement to be injected little by little into the bone at a predetermined high pressure, since bone cement has usually very high viscosity and also there is a very large pressure drop inside the bone due to a spongy substance or the like.

By sliding the slide hole of the main gasket and pressing the second pusher thereinto in a state of fixing the pressure portion cylinder and the first pusher each other in the axial direction, it is possible to generate a high pressure through aforesaid fluid in the pressure generating chamber and to make the sub gasket progress. Consequently, it is possible to eject the medicine with a high pressure from the medicine ejection unit by a proper amount for every time and to inject the medicine into the inside of the non-injection space.

Also, after the ejection, by making the main gasket inside the pressure generating chamber progress by shortening the space between the pressure portion cylinder and the first pusher through the engagement mechanism, the second pusher is returned to the original position and the pressure inside the system is reliably released simultaneously with the completion of the preparation of the next ejection process. Therefore, it is possible to prevent the pressure, which is generated inside the medicine ejection unit or inside the pressure generation unit by the pressing-in of the second pusher, from acting on a steady basis and it is possible to prevent an erroneous medicine ejection on an occasion such as operation standby or the like.

According to another aspect, a medicine injection device comprises a medicine ejection unit in combination with a pressure generation unit which generates fluid pressure. The medicine ejection unit comprises: a tubular body possessing an interior partitioned by a sub-gasket slidably arranged in a liquid-tight manner in the interior of the tubular body into a working chamber at a proximal portion of the tubular body and an action chamber distal of the working chamber at a distal end of the tubular body, a fluid flow-in port in fluid communication with the working chamber and connectable to the pressure generation unit to introduce the fluid pressure produced by the pressure generation unit into the working chamber; and a medicine ejection port at a distal end of the tubular body and in fluid communication with the action chamber, the action chamber being configured to receive medicine. The pressure generation unit comprises: a pressure portion cylinder possessing an interior in which is slidably positioned in a liquid-tight manner a main gasket defining a pressure generating chamber on a distal side of the main gasket; a plunger comprised of first and second coaxial tubular portions spaced apart from one another to define an annular space, the pressure portion cylinder being located in the annular space between the first and second tubular portion so that the first tubular portion is positioned inside the pressure portion cylinder, the first tubular portion possessing a distal end to which the main gasket is fixed so that axial movement of the pressure portion cylinder relative to the first tubular portion causes the pressure portion cylinder to axially move relative to the main gasket; and n elongated rod extending inside the first tubular portion and slidably positioned in a liquid-tight manner in a through hole in the main gasket to be moved in a distal direction to generate the fluid pressure which, upon entering the working chamber of the medicine ejection unit, urges the sub-gasket in a distal direction. The pressure portion cylinder and the plunger each possess circumferentially limited ribs which engage one another in a first relative rotational position of the pressure portion cylinder and the plunger to prevent relative axial movement between the pressure portion cylinder and the plunger and which disengage one another in a second relative rotational position of the pressure portion cylinder and the plunger to permit relative axial movement between the pressure portion cylinder and the plunger.

According to a further aspect, a method of injecting medicine comprises: positioning a medicine ejection unit relative to an ejection site, the medicine ejection unit comprising: a tubular body possessing an interior partitioned by a sub-gasket slidably arranged in a liquid-tight manner in the interior of the tubular body into a working chamber at a proximal portion of the tubular body and an action chamber at a distal portion of the tubular body which contains medicine; the working chamber being in fluid communication with a pressure generating chamber of a pressure generation unit, with an incompressible liquid in the working chamber and the pressure generating chamber; the pressure generation unit comprising: a pressure portion cylinder inside the pressure generating chamber; a first pusher which includes a main gasket slidably disposed in a liquid-tight manner in the pressure portion cylinder and defining an inner volume of the pressure generating chamber; and a second pusher passing through the first pusher and concurrently slidably positioned in a liquid-tight manner in a through hole of the main gasket; relatively rotating the pressure portion cylinder and the first pusher to a fixation position in which the pressure portion cylinder and the first pusher fixed against relative axial movement; and axially moving the second pusher toward the pressure generating chamber while the pressure portion cylinder and the first pusher are in the fixation position to pressurize the incompressible liquid in the pressure generating chamber and cause the pressurized incompressible liquid to act on the sub-gasket through the working chamber to move the sub-gasket in a distal direction and cause ejection of the medicine from the action chamber to the ejection site. The method further involves relatively rotating the pressure portion cylinder and the first pusher to a fixation release position in which the pressure portion cylinder and the first pusher fixed are relatively axially movable; relatively axially moving the pressure portion cylinder and the first pusher; relatively rotating the pressure portion cylinder and the first pusher to the fixation position to fix the pressure portion cylinder and the first pusher against relative axial movement; and axially moving the second pusher toward the pressure generating chamber while the pressure portion cylinder and the first pusher are in the fixation position to pressurize the incompressible liquid in the pressure generating chamber and cause the pressurized incompressible liquid to act on the sub-gasket through the working chamber to move the sub-gasket in a distal direction and cause the ejection of additional medicine from the action chamber to the ejection site.

### BRIEF DESCRIPTION OF DRAWINGS

FIG. 1 is a perspective view of a medicine injection device disclosed here by way of example.

FIG. 2 is a partially cross-sectional side view of the medicine injection device shown in FIG. 1.

FIG. 3 is an enlarged cross-sectional exploded side view of a main portion of a pressure generation unit constituting the medicine injection device shown in FIG. 1.

FIG. 4 is a side cross-sectional view showing a state in which respective elements of the pressure generation unit are assembled from the state shown in FIG. 3.

FIG. 5 is a cross-sectional view along the section line V-V in FIG. 3.

FIG. 6 is a cross-sectional view along the section line VI-VI in FIG. 3.

FIG. 7A is a cross-sectional view along the section line VII-VII in FIG. 4 in a state in which a first rib is arranged in a rib path.

FIG. 7B is a cross-sectional view taken along the VII-VII section line showing the first rib engaged with a rib set by rotating a pressure portion cylinder from the state shown in FIG. 7A.

FIG. 8 is an operation flow relating to a medicine ejection by the medicine injection device shown in FIG. 1.

FIG. 9 is an enlarged cross-sectional exploded side view of a main portion of a pressure generation unit constituting the medicine injection device according to a second embodiment disclosed here by way of example.

FIG. 10 is a cross-sectional view along the section line X-X in FIG. 9 in a state in which the pressure portion cylinder is inserted into the inside of a tubular space of a first pusher and the first rib is arranged in the rib path.

FIG. 11A is a cross-sectional view in a state in which the pressure portion cylinder is rotated counterclockwise from the state shown in FIG. 10.

FIG. 11B is a cross-sectional view in a state in which the pressure portion cylinder is rotated clockwise from the state shown in FIG. 11A.

FIG. 12 is a schematic explanatory view in which the pressure portion cylinder and the first pusher shown in FIG. 10 are developed by 360° in the circumferential direction in order to explain an engagement operation between the first rib and the rib set in the medicine injection device shown in FIG. 9.

FIG. 13 is a partially omitted cross-sectional side view showing a medicine ejection unit according to a modified example.

### DETAILED DESCRIPTION

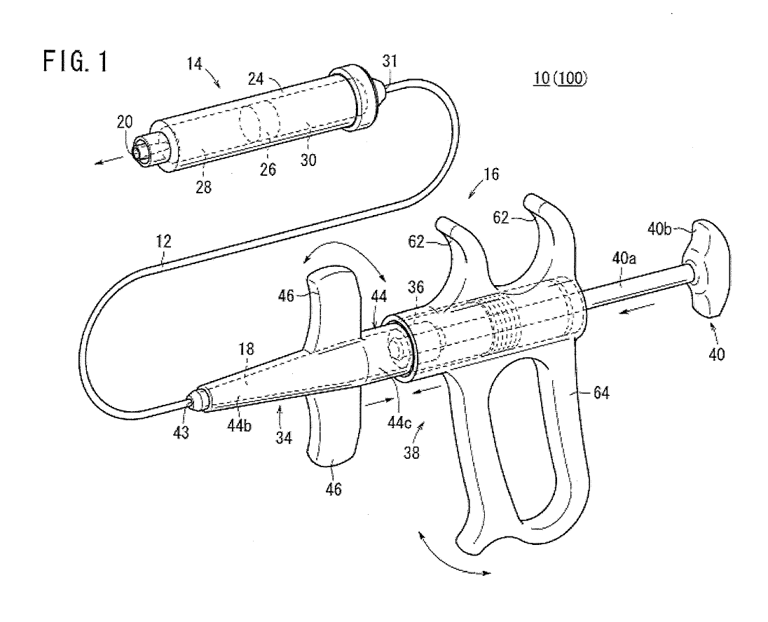

Referring to FIG. 1, the medicine injection device 10 disclosed here includes a medicine ejection unit 14 coupled to the distal side of a long flexible tube 12 and a pressure generation unit 16 coupled to the proximal side of the flexible tube 12. The medicine injection device 10 is an instrument for ejecting a predetermined medicine (filling member, injection member) from the medicine ejection unit 14 by pressure generated in a pressure generating chamber 18 of a pressure generation unit 16 through a predetermined liquid 19 and for injecting it into a desired space. As an example, the medicine injection device 10 is used to inject bone cement into a bone in percutaneous vertebroplasty. The aforementioned medicine can be in the form of, for example, a bone cement such as calcium phosphate based bone cement (CPC), polymethyl methacrylate (PMMA) based bone cement or the like. It is also possible to use granules formed of an inorganic material such as calcium phosphate based ceramics, alumina ceramics, zirconia ceramics, titanium or the like.

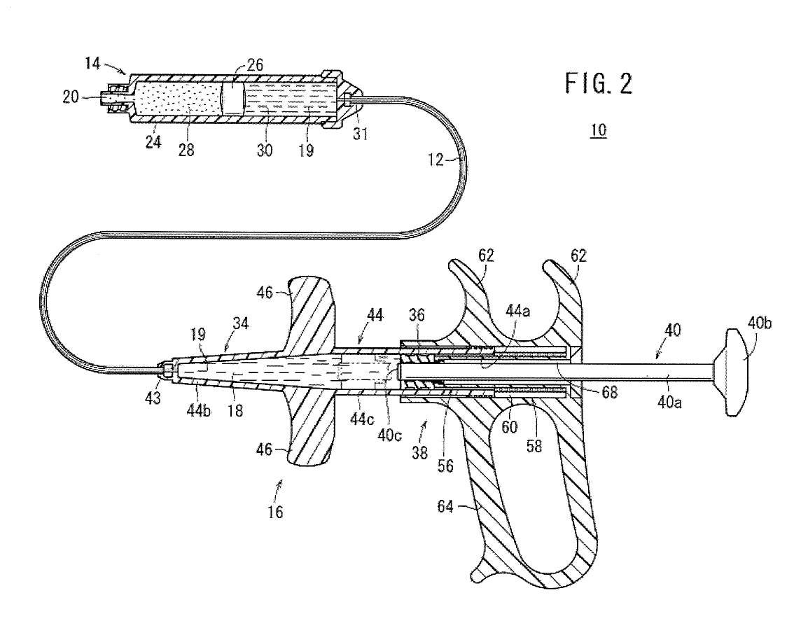

In FIG. 1 and FIG. 2, the medicine ejection unit 14 side of the flexible tube 12 is referred to as the “distal end” side, the pressure generation unit 16 side of the flexible tube 12 is referred to as the “proximal end (rear end)” side. Similar references are used in the other drawings.

The medicine ejection unit 14 has a tubular body (cylindrical body) 24 provided with an ejection port (medicine ejection port) 20 at its distal end, and a floating gasket (sub gasket) 26 which is slidable in a liquid-tight state inside the tubular body 24. The inside of the tubular body 24 is partitioned by the floating gasket 26 into an action chamber 28 located on the distal side in the axial direction and a working chamber 30 located on the proximal side and serving as the flow-in side or introduction side for the liquid 19. The ejection port 20 is a luer connector and, when a medicine is injected into a bone, a needle or the like for bone cement, can be coupled to the luer.

Because the action chamber 28 is located between the ejection port 20 and the floating gasket 26, the action chamber 28 is able to store the medicine and supply the medicine to the ejection port 20 along with progression in the direction of the distal end of the floating gasket 26.

The working chamber 30 fluidly communicates with the flexible tube 12 by a flow-in port 31 for the liquid 19 (see FIG. 2) located on the proximal side of the tube body 24. Thus, the pressure generated through the liquid 19 at the pressure generating chamber 18 of the pressure generation unit 16 acts on the working chamber 30 through the flexible tube 12 and makes the floating gasket 26 progress (i.e., moves the floating gasket 26). More specifically, by virtue of the flowing liquid 19, which fills the interiors of the working chamber 30, the flexible tube 12 and the pressure generating chamber 18, from the pressure generating chamber 18 through the flexible tube 12 into the working chamber 30 by way of the flow-in port 31, the floating gasket 26 moves. The liquid 19 is an incompressible fluid (for example water) such that it serves as a motive force with which to make the floating gasket 26 progress or move distally without losing the pressure generated at the pressure generating chamber 18 as much as possible, and gaseous components with high compression ratios, such as air and the like, are excluded as much as possible.

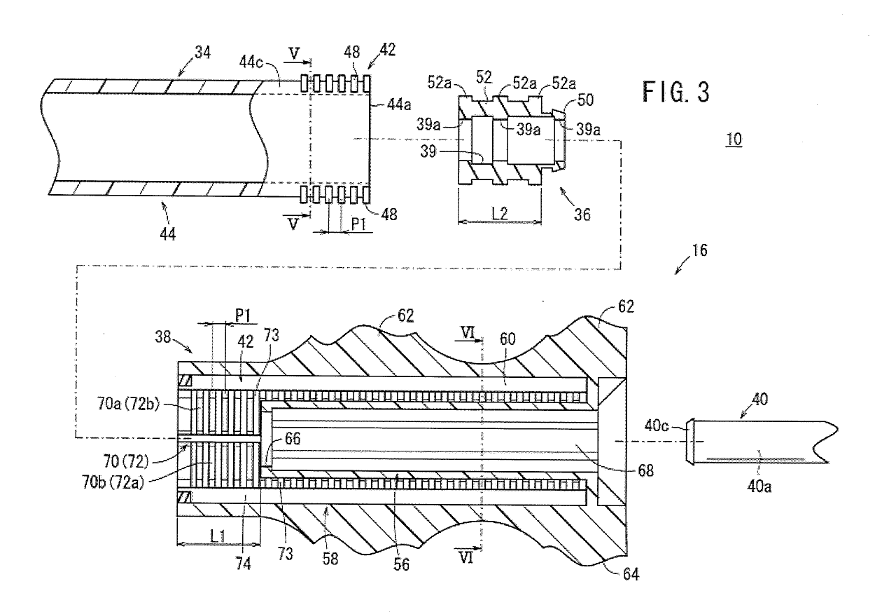

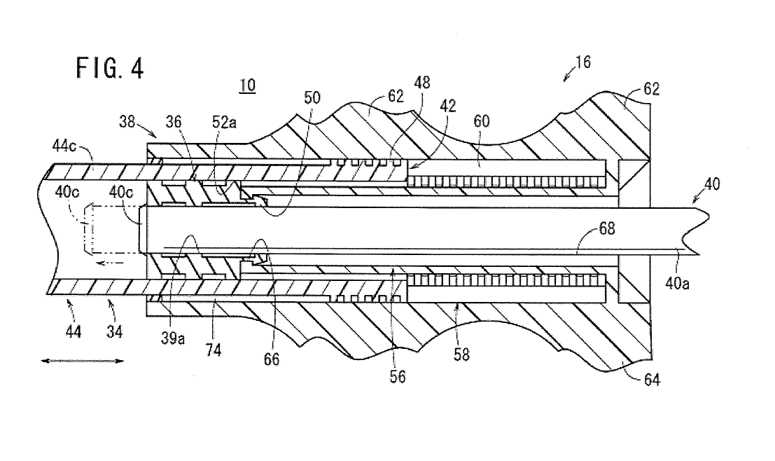

The pressure generation unit 16 includes a pressure portion cylinder 34 coupled at its distal end to the flexible tube 12, a hollow gasket (main gasket) 36 which defines the inner volume of the pressure generating chamber 18 by sliding in a liquid-tight state inside the pressure portion cylinder 34, a first pusher (first plunger) 38 having a double tube structure, to which the hollow gasket 36 is fixed on the distal side thereof, and a second pusher (second plunger) 40 provided with a rod 40a which passes-through the inside of the first pusher 38 from the proximal side to the distal side and which slides in a liquid-tight state with respect to a slide hole 39 (see FIG. 3 and FIG. 4) passing through in the sliding direction of the hollow gasket 36. For the pressure portion cylinder 34 and the first pusher 38, the coupling position thereof in the axial direction is changeable appropriately by of an engagement mechanism 42 (see FIG. 3 and FIG. 4) which allows mutual fixation and fixation release for every predetermined pitch in the axial direction. Details about this will be described later.

The pressure portion cylinder 34 includes a tubular portion or tubular member 44 which is coupled with the flexible tube 12 at an outlet port 43 at the distal end. The pressure generating chamber 18 is located in the pressure portion cylinder 34 and is formed by a mechanism in which the hollow gasket 36 is inserted from an opening end 44a at the proximal end. The pressure portion cylinder 34 includes a pair of wings (first operation member) 46, 46 having flat plate shapes, which project from the outer circumferential surface of the tubular portion 44 toward the outer diameter direction (radially outwardly) and which are somewhat curved toward the distal side.

The tubular portion 44 has a taper region 44b in the shape of a circular truncated cone formed on the side more distal than the approximate center in the axial direction, and a cylindrical region 44c whose outer diameter and inner diameter are formed approximately uniformly and at the proximal end of which an opening end 44a is formed. The inner diameter of the cylinder region 44c is, for example, set at around 4 mm to 16 mm and the hollow gasket 36 axially slides in a liquid-tight state on the inner circumferential surface of the cylinder region 44c. In this case, the inner diameter of the cylinder region 44c can be the same as or different from the inner diameter of the tube body 24 of the medicine ejection unit 14, but the inner volume of the pressure generating chamber 18 formed inside the tubular portion 44 is equal to or greater than the inner volume of the action chamber 28 of the medicine ejection unit 14.

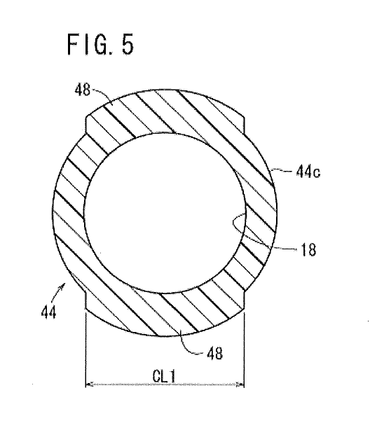

As shown in FIG. 3 and FIG. 5, a pair of first ribs 48, 48 is formed on the outer circumferential surface at the proximal side of the tubular portion 44 (cylinder region 44c). The pair of first ribs 48, 48 are configured as a portion of the tubular portion 44 expanded outwardly to have an enlarged outer diameter relative to circumferentially adjacent portions. The first ribs 48 are thin plate-shaped members spaced apart from another in the axial direction of the tubular portion 44. Each of the first ribs 48 is circumferentially limited, meaning each rib extends over only a part of the circumferential extent (360°) of the tubular portion 44 so that each rib does not extend around the entire 360° extent of the tubular portion 44. A plurality of the first ribs 48 (in this embodiment disclosed by way of example, 6 pieces) are arranged in the axial direction at a pitch P1 in which the interval is approximately the same as or somewhat wider than the plate thickness of the first ribs 48.

The hollow gasket 36 is formed, for example, by a flexible member such as rubber, silicone or the like. As shown in FIG. 3, the hollow gasket 36 includes an attachment portion 50 having a smaller diameter on the proximal side and a seal portion 52 having a larger diameter than that of the attachment portion 50. A seal ring portion 52a is provided on the outer circumferential surface of the seal portion 52. In this illustrated example, the seal ring portion 52a is in the form of a plurality of axially spaced apart diameter-expanded (outwardly enlarged) pieces. The outer diameter of the seal ring portion 52a (seal portion 52) is approximately the same as or somewhat larger than the inner diameter of the tubular portion 44 (cylinder region 44c). Thus, the seal ring portion 52a (seal portion 52) functions as a liquid-tight lip seal which is closely held against the inner circumferential surface of the tubular portion 44 in a slidable manner.

An axially extending slide hole 39 passes through the hollow gasket 36. The rod 40a of the second pusher 40 passes slidably through the slide hole 39. On the inner circumferential surface of the slide hole 39, there is provided a seal ring portion 39a in which a plurality of axially spaced apart diameter-reduced portions are positioned parallel to one another. The inner diameter of the seal ring portion 39a (slide hole 39) is approximately the same as or somewhat smaller than the outer diameter of the rod 40a of the second pusher 40. Thus, the seal ring portion 39a (slide hole 39) functions as a liquid-tight lip seal which is closely held against or closely engages the rod 40a in a slidable manner.

As shown in FIG. 2 to FIG. 4, the first pusher 38 includes a first tubular portion 56 positioned inside the tubular portion 44 (pressure generating chamber 18) with the hollow gasket 36 fixed at the distal end and a second tubular portion 58 which together form a double tube structure in which the two tubular portions 56, 58 are coaxially arranged with a predetermined space between the two in the outer diameter direction of the first tubular portion 56. The gap between the first tubular portion 56 and the second tubular portion 58 functions as a tubular space 60 through which the tubular portion 44 (cylindrical region 44c) passes. In the illustrated embodiment, the first tubular portion 56 is a cantilevered tubular portion.

As shown in FIG. 1 and FIG. 2, on the outer circumferential surface of the second tubular portion 58, there are formed a pair of finger hook portions 62, 62 which are parallel to one another and project toward the outer diameter direction and which are in the shape of flat plates curved toward the distal side. The outer circumferential surface of the second tubular portion 58 also includes a rectangular ring shaped finger hook ring 64 which has a flat plate shape projecting from a position approximately 180° circumferentially spaced on the opposite side of the finger hook portion 62 toward the outer diameter direction (see FIG. 1 and FIG. 2). The finger hook portion 62 and the finger hook ring 64 are regions to which the operator hooks his fingers together with the wing 46 of the pressure portion cylinder 34 when pulling-in the pressure portion cylinder 34 and the first pusher 38 so as to be close to each other.

As shown in FIG. 3 and FIG. 4, the first tubular portion 56 has an attachment hole 66 at its distal end, to which a proximal end diameter-expanded portion of the attachment portion 50 of the hollow gasket 36 is fitted by insertion and fixed, and a guide hole 68 opened on the proximal side thereof.

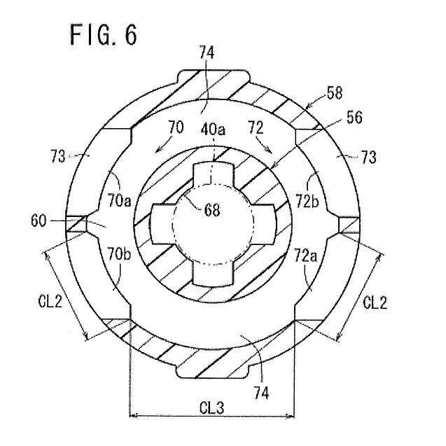

The guide hole 68 passes-through in the axial direction from the proximal end of the first tubular portion 56 to the attachment hole 66 of the distal end and allows the rod 40a of the second pusher 40 to pass through to the slide hole 39 of the hollow gasket 36 and concurrently guides the axial sliding of the second pusher. As shown in FIG. 6, the guide hole 68 has a cross-shaped configuration in cross-section with a center portion of the hole portion having a circular configuration along the axial direction to guide the rod 40a slidably. The circular inner circumferential surface is interrupted at four places so that the four circumferentially spaced apart portions of the inner surface of the guide hole 68 contacting the rod 40a have approximately trapezoidal cross-sections, are arranged at 90° intervals and are elongated in the axial direction. In this manner, the guide hole 68 guides the rod 40a by 4-point support, so that it is possible to perform stable guiding in the axial direction. It is also possible to appropriately reduce the slide resistance with respect to the rod 40a. It is also possible to configure the guide hole 68 as a simple circle which slides on the full circumferential surface of the rod 40a.

As shown in FIG. 3 and FIG. 4, the second tubular portion 58 is configured such that the proximal end is closed by being coupled with the outer circumferential surface of the proximal end of the first tubular portion 56. The tubular portion 44 of the pressure portion cylinder 34 is insertable from the opened distal side to the inside of the tubular space 60. Therefore, the inner diameter of the second tubular portion 58 is a diameter approximately the same as or somewhat larger than the maximum outer diameter (outer diameter of a pair of first ribs 48, 48) of the tubular portion 44.

As shown in FIG. 3, the length L1 in the axial direction from the distal end of the second tubular portion 58 to the distal end of the first tubular portion 56 is approximately the same as the length L2 in the axial direction of the attachment portion 50 of the hollow gasket 36. Thus, as shown in FIG. 4, when the hollow gasket 36 is positioned in the attachment hole 66, the distal end surface of the hollow gasket 36 is approximately flush with the distal end surface of the second tubular portion 58.

As shown in FIG. 3 and FIG. 6, the second tubular portion 58 is formed with a pair of rib sets (second ribs) 70, 72 which are configured as diameter-reducing & projecting portions of the inner circumferential surface of the second tubular portion 58 which project inwardly (radially inward) and which are engageable with the first ribs 48 of the pressure portion cylinder 34. The rib sets 70, 72 are thin plate-shaped members in the axial direction of the second tubular portion 58 and are arranged plural in number and spaced apart axially over the second tubular portion 58 with a pitch P1 that is approximately the same as or somewhat wider than the plate thickness thereof. More specifically, the rib sets 70, 72 are arranged or spaced apart in the axial direction by a pitch P1 identical with that of the first ribs 48. The number of rib sets 70, 72 is more than that of the first ribs 48 and they are arranged over the axial extent of the tubular space 60 so as to be engageable with each other even in a state in which the tubular portion 44 is inserted to the maximum extent inside the tubular space 60. Each of the second ribs is circumferentially limited, meaning each rib extends over only a part of the circumferential extent (360°) of the second tubular portion 58 so that each rib does not extend around the entire 360° extent of the second tubular portion 58.

As shown in FIG. 3 and FIG. 6, the respective ribs sets 70, 72 include first small ribs 70a, 72a and second small ribs 70b, 72b which are respectively aligned in the circumferential direction. For the first small ribs 70a, 72a and the second small ribs 70b, 72b, the length (width) CL2 in the circumferential direction is around half of the length CL1 of the first rib 48 in the circumferential direction (see FIG. 5). The ribs constituting the respective rib sets 70, 72 (for example, first small rib 70a and second small rib 70b) are aligned by spacing a small gap. That is, there is a clearance between the first small rib 70a and the second small rib 70b as generally shown in FIG. 6. Also, the space CL3 in the circumferential direction between the rib set 70 and the rib set 72 is somewhat larger than the length CL1 of the first rib and depending on this configuration, it is possible for the first rib 48 to pass through between the rib sets 70, 72 as a rib path 74 in the axial direction.

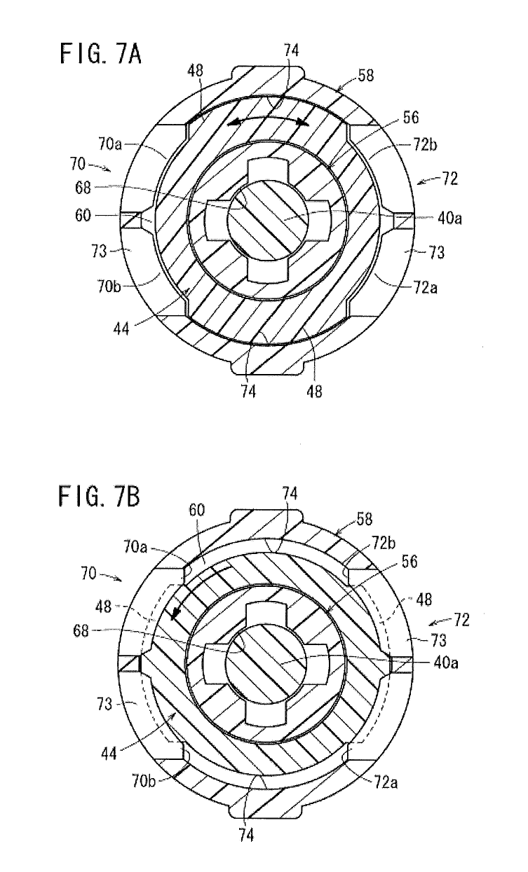

Therefore, by adjusting the rotation direction of the pressure portion cylinder 34 and by setting the position such that the first rib 48 corresponds to or is positioned in the rib path 74 as shown in FIG. 7A in a state in which the tubular portion 44 of the pressure portion cylinder 34 is inserted into the tubular space 60 of the first pusher 38 (see FIG. 4), the tubular portion 44 is moved relatively smoothly in the axial direction of the tubular space 60, and it is possible, as shown by broken lines in FIG. 2, to slide the hollow gasket 36 inside the pressure generating chamber 18. On the other hand, as shown in FIG. 7B, by rotating the pressure portion cylinder 34 counterclockwise (or clockwise) approximately 90° in which the respective first ribs 48 correspond to or face the respective rib set 70, 72 and by engaging the pressure portion cylinder 34 between the rib sets 70 and between the rib sets 72 which are aligned in the axial direction, the positions of the pressure portion cylinder 34 and the first pusher 38 in the axial direction are fixed.

In this manner, the first ribs 48, rib sets 70, 72 (first small ribs 70a, 72a and second small ribs 70b, 72b) make both the pressure portion cylinder 34 and the first pusher 38 carry out fixation and fixation release for every predetermined pitch P1 in the axial direction thereof, and they function as an engagement mechanism 42 which changeably constitutes the fixation position in the axial direction.

As understood from FIG. 3 and FIG. 6, at axially spaced portions of the respective first small ribs 70a, 72a and the respective second small ribs 70b, 72b aligning in the axial direction of the second tubular portion 58, there are provided hole portions 73 communicating with inner and outer surfaces of the second tubular portion 58. It is possible for these first small ribs 70a, 72a and the respective second small ribs 70b, 72b to be relatively easily manufactured by a die having a structure in which protrusions provided in right and left split molds abut a die (center pin) for molding the guide hole 68, for example, when molding the first pusher 38 (second tubular portion 58) by injection mold, and the hole portions 73 are formed by the protrusions.

As shown in FIG. 1 to FIG. 4, the second pusher 40 includes an elongated and narrow-diameter rod 40a and a press-in portion 40b which serves as a operation (steering) portion when the rod 40a is pushed into the pressure generating chamber 18 of the pressure portion cylinder 34. The outer diameter of the rod 40a is, for example, around 2 mm to 6 mm.

As shown in FIG. 3 and FIG. 4, at the distal end of the rod 40a, there is provided a distal end head portion 40c which is diameter-expanded (outwardly enlarged) to posses a flange shape. The distal end head portion 40c hooks on the distal side opening portion of the slide hole 39 of the hollow gasket 36, thereby inhibiting or preventing the pulling-out of the rod 40a from the hollow gasket 36. When the hollow gasket 36 is formed as a flexible member such as rubber or the like as described above, it is possible for the distal end head portion 40c having a somewhat larger diameter compared with that of the slide hole 39 (seal ring portion 39a) to pass-through the inside of the slide hole 54 relatively easily at the time of assembly.

With respect to the medicine ejection unit 14 or the pressure generation unit 16, it is preferable to form the tube body 24, the first pusher 38, the second pusher 40 or the like from, for example, a polyolefin such as polypropylene, polyethylene, cyclic polyolefin, polymethylpentene 1 or the like, a resinous material such as polyester, nylon, polycarbonate, polyethersulphone or the like, or a metal material such as stainless steel or the like, and it is preferable if the floating gasket 26 or the hollow gasket 36 is formed of, for example, an olefin-based elastomer, a vulcanized rubber such as silicone rubber, butyl rubber, fluorine rubber or the like, and further, materials made by coating those with fluorine resin, or the like. The flexible tube 12 can possess a construction in which a thread of Kevlar, nylon, polyphenylene sulfide, stainless steel or the like is wound on the tube wall inside or the outer surface of the comparatively flexible polyolefin in a mesh or coil shape. This imparts desired pressure resistance to the flexible tube 12. It is also possible to employ a multi-layered tube in which polypropylene or fluorine resin having water resistance is arranged only on the inner surface.

These respective components are manufacturable by injection molding, extrusion molding, press molding or the like. Connection of the respective components is possible by adhesion, using an adhesive agent or thermal fusion. A connection method such as a caulking tool, screw engagement or the like is possible to connect the respective cylinders and the tube.

The description which follows will explain the operations and actions of the medicine injection device 10 according to the embodiment described above as an example of the medicine injection device.

In the case of using the medicine injection device 10 in the percutaneous vertebroplasty, as an example, the inside of the action chamber 28 of the medicine ejection unit 14 is filled with a predetermined amount (for example, 5 ml) of the medicine (bone cement of CPC, PMMA or the like) beforehand. This filling is carried out by, for example, a method in which the first pusher 38 is pressed-in with respect to the pressure portion cylinder 34 and in a state in which the hollow gasket 36 is pressed into the pressure generating chamber 18 to the maximum extent, a distal end tube coupled to the ejection port 20, or the ejection port 20, is inserted into the container filled with bone cement which is medicine and the medicine is sucked inside the action chamber 28 by pulling the first pusher 38, or a method in which a syringe is coupled to the ejection port 20 and the syringe is pressurized to fill the inside of the action chamber 28 with the medicine. Next, an ejection rod (needle for bone cement) or the like is connected to the ejection port 20 of the medicine ejection unit 14, and the filling of the medicine is executed under X-ray illumination.

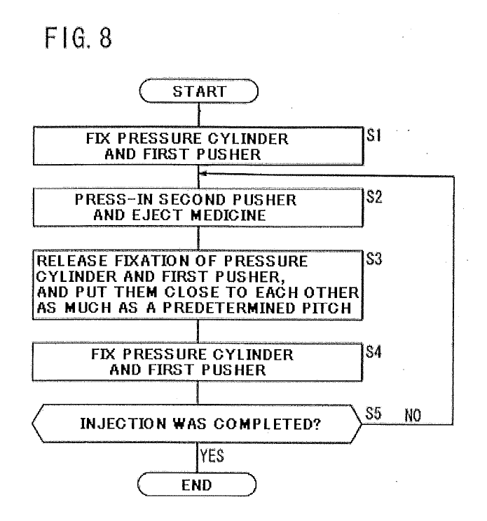

The operation procedure involving the medicine ejection by the medicine injection device 10 is shown in FIG. 8.

In order to eject the medicine inside the action chamber 28 from the ejection port 20, both the pressure portion cylinder 34 and the first pusher 38 are first fixed in an appropriate initial position corresponding to the amount of injection of the medicine (for example, 5 ml) by the first rib 48 and the rib sets 70, 72 of the engagement mechanism 42 (step S1). That is, by rotating the pressure portion cylinder 34 in which the respective first ribs 48 correspond to or face the respective rib set 70, 72 and by engaging the pressure portion cylinder 34 between the rib sets 70 and between the rib sets 72 which are aligned in the axial direction, the positions of the pressure portion cylinder 34 and the first pusher 38 in the axial direction are fixed as generally shown in FIG. 7. Next, the press-in portion 40b is pressed-in toward the distal end direction by a user's palm or the like from a state in which the second pusher 40 is pulled, to the maximum extent, in the proximal direction (see FIG. 2) so as to make the rod 40a progress or move distally forward. As shown by the broken-line outline in FIG. 4, pressing the press-in portion 40b in the distal direction causes the distal end head portion 40c to move into the pressure generating chamber 18 (step S2). More specifically, both the pressure portion cylinder 34 and the first pusher 38 are fixed in the axial direction and the hollow gasket 36 is also fixed by the first pusher 38, so that the rod 40a is pressed into the pressure generating chamber 18 while sliding relative to the hollow gasket 38 in the slide hole 39 of the hollow gasket 36.

Thus, a pressure corresponding to the reduced inner volume (void inner volume) of the liquid 19, resulting from the forward movement of rod 40a inside the pressure generating chamber 18, is generated inside the pressure generating chamber 18. Concurrently, this pressure causes the liquid 19 composed of the incompressible fluid (for example, water) filling the interior of the pressure generating chamber 18 and the flexible tube 12 to flow from the flow-in port 31 into the working chamber 30 and thus, the floating gasket 26 is pressed or forwardly moved from the working chamber 30 side toward the action chamber 28 side. At that time, the liquid 19 is substantially not compressed. Moreover, the outer diameter (for example, 5 mm) of the rod 40a of the second pusher 40 is a relatively narrow-diameter, so that it is possible to generate a relatively high pressure of 100 atmospheres (about 100 Mpa) or more inside the chamber 18 even by pushing with a human hand. Therefore, the high pressure generated in the pressure generating chamber 18 is not absorbed by the liquid 19, but is instead directly transferred to and directly acts upon the floating gasket 26. More specifically, the floating gasket 26 progresses or moves forward by an amount corresponding to the void inner volume of the rod 40a, and the appropriate amount (for example, 1 ml) of the medicine corresponding to the aforesaid void inner volume is reliably ejected from the ejection port 20 under the high pressure. In other words, by sealing the aforesaid liquid 19 in the working chamber 30, the aforesaid liquid 19 functions as a connector (piston rod) mechanically coupling the rod 40a with the floating gasket 26, and the press-in pressure by the second pusher 40 causes ejection of the medicine directly.

Subsequently, the pressure portion cylinder 34 is rotated with respect to the first pusher 38 by rotating the wing 46, and the engagement state between the first rib 48 and the rib sets 70, 72, which constitute the engagement mechanism 42, is thereby released (see FIG. 7A), and the first rib 48 is movable in the rib path 74. Thus, the pressure portion cylinder 34 and the first pusher 38 approach each other (i.e., the extent of axial overlap of the pressure portion cylinder 34 and the first pusher 38 increases), for example by an amount corresponding to one pitch or a few pitches based on the pitch P1 of the first rib 48 and the rib sets 70, 72, more specifically by an amount up to that which corresponds to the void inner volume of the second pusher 40 (step S3).

In that case, the hollow gasket 36 (first tubular portion 56) progresses into the pressure generating chamber 18 or moves in the distal direction so that the liquid 19 is pushed into the pressure generating chamber 18 and a certain degree of pressure is generated. But the diameter (for example, 12 mm) of the hollow gasket 36 is considerably larger than the diameter (for example, 5 mm) of the rod 40a. It is difficult to generate, by hand, high pressure such as that associated with the above-described human-hand movement of the rod 40a and so the floating gasket 26 also does not progress. Ultimately, a force acts in the direction causing the rod 40a of the second pusher 40 to return toward the original position by virtue of the liquid 19 in the pressure generating chamber 18, and the second pusher retreats as much as the volume corresponding to the press-in amount in step S2 until the distal end head portion 40c abuts onto the opening end of the slide hole 39 of the hollow gasket 36. Consequently, a state is reached in which the whole pressure generated by the progress of the second pusher 40 and the hollow gasket 36 is released. The generating pressure corresponding to the void inner volume of the second pusher 40 is used to eject the medicine and so it is possible to eject the appropriate amount of medicine (for example, 1 ml) at the high pressure. If the pushing force applied to the second pusher 40 is stopped after ejection, the pressure inside the system is released reliably.

Therefore, again, by fixing the pressure portion cylinder 34 and the first pusher 38 by the engagement mechanism 42 (step S4), the hollow gasket 36 progresses as much as a predetermined pitch (as much as void inner volume by second pusher 40) of the engagement mechanism 42 toward the pressure generating chamber 18 and the floating gasket 26 also progresses as much as the volume corresponding to the void inner volume of the second pusher 40 of the hollow gasket 36. Other than that, it becomes an approximately similar state as the initial state before the injection start shown in FIG. 2.

In step S5, the operations of step S1 to step S4 described above are repeated until a necessary and sufficient amount of the medicine is injected into the bone (NO in step S5), and after the injection of the necessary and sufficient amount of the medicine is completed (YES in step S5), the ejection port 20 of the medicine ejection unit 14 is removed to the outside of the body, thereby making it possible to execute the percutaneous vertebroplasty relatively easily.

The medicine injection device 10 according to this embodiment disclosed by way of example reliably ejects a constant amount (for example, 1 ml) of the medicine every one time of ejection (step S1 to S4) under a predetermined high pressure. Moreover, the pressure inside the system of the medicine injection device 10 is released during the intervening time interval following completion of one ejection process until the next ejection process. In other words, the fact that the medicine injection device 10 is constituted as a two-stage piston type injection device provided with the second pusher 40 and the hollow gasket 36, the pressure inside the system is released at the moment of releasing the hand after the second pusher 40 is pressed-in and thereafter pressure is not generated by forward movement of the hollow gasket 36.

Thus, for example, it is possible to reliably prevent a high pressure state of around 100 atmospheres from excessively acting on the flexible tube 12 and the floating gasket 26, and it is possible to prevent erroneous medicine ejection during the operational standby while also preventing a load at the connection portion with the flexible tube 12. Also, even in a case in which, for example, it is necessary to inject 4 ml into the inside of the bone despite a previous plan to inject 5 ml of medicine (5 ejection processes each ejecting 1 ml of medicine) into the inside of the bone, pressure is not generated on the liquid 19 inside the medicine injection device 10 at the point in time when the ejection process is finished, so that the medicine is not erroneously ejected from the ejection port 20 and it is possible to remove the medicine ejection unit 14 from the patient.

It is possible to adjust the coupling position or engaging position of the pressure portion cylinder 34 and the first pusher 38 in the axial direction according to the pitch P1 of the first rib 48 and the rib sets 70, 72 by a relatively simple operation in which the cylinder 34 and the pusher 38 are rotated relative to each other and are shifted in the axial direction, with the second pusher 40 being reliably returned to the initial position at the time of the above-described adjustment. Consequently, after ejection of the medicine by the second pusher 40, it is possible to set the pressure portion cylinder 34, the first pusher 38 and the second pusher 40 to the position (initial position) corresponding to the next ejection process.

In the medicine injection device 10, it is sufficient that the hollow gasket is a one piece hollow gasket 36 which slides at the pressure generation unit 16. Also the engagement mechanism 42 between the pressure portion cylinder 34 and the first pusher 38 is a construction in which only the ribs are provided. Further, the second pusher 40 enables pressure to be generated by virtue of it simply passing through the inside of the first pusher 38 and being pressed-in, and after ejection of the medicine, the second pusher 40 is returned to the initial position by simply moving the pressure portion cylinder 34 and the first pusher 38. In this manner, the structure of the medicine injection device 10 is relatively simple and low-cost, and concurrently the injection operation is relatively simply.

It is necessary for the aforesaid pitch P1, which serves as the standard of the movement amount of the hollow gasket 36 after the ejection, to be set such that as much as the maximum void inner volume by the second pusher 40 is absorbable. In other words, it is necessary to be able to fix the pressure portion cylinder 34 and the first pusher 38 by making the hollow gasket 36 progress for just as much as the void inner volume by the second pusher 40. Therefore, the void inner volume V1 corresponding to 1 pitch of the pitch P1 of the hollow gasket 36 is set within 1 time or preferably around 0.2 times to 1 time (V1=0.2V2 to V2) with respect to the maximum void inner volume V2 pressable by the second pusher 40 in a state in which the pressure portion cylinder 34 and the first pusher 38 are fixed. Thus, after the pressing-in of the second pusher 40, it becomes possible for the pressure portion cylinder 34 and the first pusher 38 to reliably approach each other by at least as much as one pitch.

Also, in a case in which the medicine injection device 10 is used for a medicine injectable at a certain degree of low pressure and for the space to be injected, the pressure portion cylinder 34 and the first pusher 38 are set to a state in which the first rib 48 corresponds to the rib path 74 and in this state, the pressure portion cylinder 34 and the first pusher 38 are pulled toward each other. In that case, a large amount of injection at one time by the hollow gasket 36 becomes possible compared with high-pressure injection in small amounts by the second pusher 40 (see broken line in FIG. 2). More specifically, the medicine injection device 10 is a two-stage piston (two-stage plunger) structure by virtue of the first pusher 38 (hollow gasket 36) and the second pusher 40, and only the hollow gasket 36 which is one of those pistons is used, and if the second pusher 40 which is the other piston is fixed while being kept at the initial position, approximately similarly to a usual syringe or the like, it is possible to use it for the medicine ejection in a large amount and also at low pressure, and versatility is relatively high. It is possible for such a use method approximately similar to a usual syringe or the like to be used effectively even when a predetermined medicine from the ejection port 20 is made to fill (sucked into) the inside of the action chamber 28 as described above.

In the medicine injection device 10, by providing the wing 46 of the pressure portion cylinder 34, and the finger hook portions 62 and the finger hook ring 64 of the first pusher 38, allowing both to be grasped with a single hand (or both hands), it is possible to rotate or pull-in the pressure portion cylinder 34 and the first pusher 38 relatively easily, and steerability is high. Also, in the medicine injection device 10, the pressure portion cylinder 34 is inserted into the first pusher 38 to the maximum extent, and by pressing-in the second pusher 40 to the maximum extent, it is compactly storable.

The medicine ejection unit 14 is connected with the pressure generation unit 16, which lies on the operation side, through the long scale flexible tube 12, so that it is possible to carry out the injection operation at a position apart from the medicine ejection unit 14 near to the X-ray tube. Therefore, if the length of the flexible tube is, for example, to be 5 cm or more, the operator's exposure to X-rays on an occasion of injection of the bone cement can be reduced. It is preferable for the length of the flexible tube to be 15 cm or more to 50 cm or less. Depending on the use condition or the like of the medicine injection device 10, it is also possible to omit the flexible tube 12 and to employ a construction in which the medicine ejection unit 14 is provided at the distal end of the pressure portion cylinder 34 in direct connection or integrally.

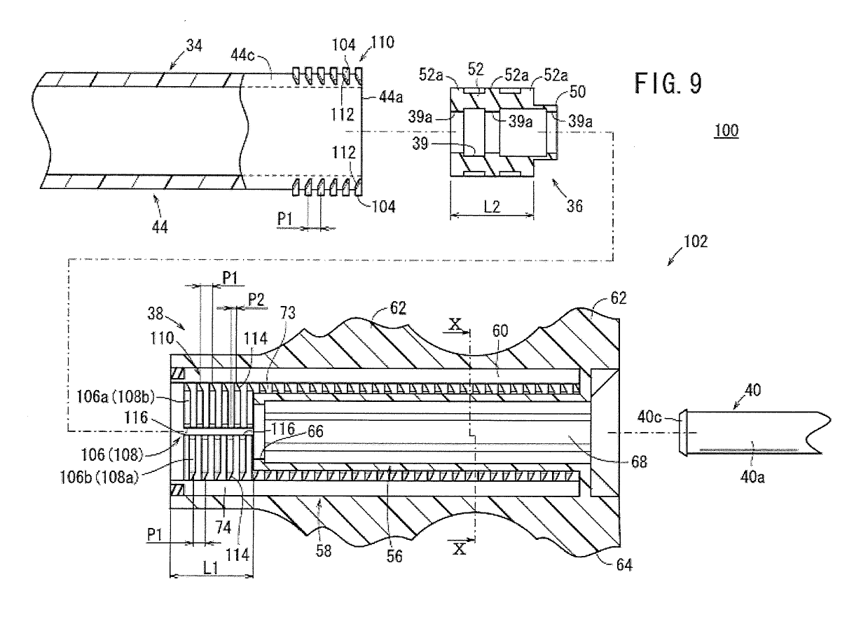

FIG. 9 illustrates a main portion of a pressure generation unit 102 constituting a medicine injection device 100 according to a second embodiment disclosed by way of example. features and aspects of this embodiment which are the same as in the earlier embodiment are identified by common reference numerals, and a detailed description of all of such features and aspects is not repeated here

The medicine injection device 100 is provided with a pressure generation unit 102 in which the construction of the pressure generation unit 16 of the medicine injection device 10 described above is changed. As shown in FIG. 9, the pressure generation unit 102 is basically similar to the pressure generation unit 16 shown in FIG. 3 except for inclusion of an engagement mechanism 110, constituted by a first rib 104 and rib sets 106, 108, instead of the engagement mechanism 42.

As shown in FIG. 9, the first rib 104 is different from the first rib 48 shown in FIG. 3 in that the first rib 104 possesses an inclination surface (first inclination surface) 112 whose width narrows toward the proximal end in the axial direction of the tubular portion 44 is formed at both end portions in the circumferential direction (longitudinal direction) thereof.

The rib sets 106, 108 include, similar to the rib sets 70, 72 shown in FIG. 3, first small ribs 106a, 108a and second small ribs 106b, 108b, which are respectively aligned in the circumferential direction. But there is a difference from the above-described rib set 70 (72) in that the positions of the first small rib 106a and the second small rib 106b (first small rib 108a and second small rib 108b) constituting each rib set 106 (108) in the axial direction of the second tubular portion 58 deviate from each other.

More specifically, in the axial direction of the second tubular portion 58, the first small ribs 106a alone and the first small ribs 108a alone (second small ribs 106b alone and second small ribs 108b alone are also similar) are aligned at the same pitch P1 as that of the first ribs 104 and the above-described rib sets 70, but the first small rib 106a and the second small rib 106b (first small rib 108a and second small rib 108b are also similar) deviate from each other by as much as a pitch P2 which is half the pitch P1. In other words, in the rib sets 106, 108, with respect to the first small rib 106a and the first small rib 108a (second small rib 106b and second small rib 108b) which face to each other, the phases thereof in the axial direction of the second tubular portion 58 are the same, and with respect to the first small rib 106a and the second small rib 106b (first small rib 108a and second small rib 108b) which adjoin each other in the circumferential direction, the phases thereof in the aforesaid axial direction deviate from each other by as much as a half pitch.

At the end portion facing the rib path 74 of such a rib set 106 (108), more specifically, at the end portions on the sides facing the rib paths 74 of the first small ribs 106a, 108a and the second small ribs 106b, 108b, there are formed inclination surfaces (second inclination surfaces) 114 whose widths narrow toward the distal side in the axial direction of the second tubular portion 58. The inclination surface 114 has a shape allowing the inclination surface 112 of the first rib 104 to get over while they slide on each other.

Therefore, in the medicine injection device 100 relating to this embodiment, for example, on an occasion when the second pusher 40 is pressed into the pressure generating chamber 18 and the medicine is ejected from the ejection port 20 and thereafter, the hollow gasket 36 progresses or moves inside the pressure generating chamber 18 by making the pressure portion cylinder 34 and the first pusher 38 close to each other, the pressure portion cylinder 34 and the first pusher 38 are rotated in opposite directions as if they are twisted oppositely to each other. In that way, it is possible for the pressure portion cylinder 34 and the first pusher 38 to approach each other by every aforesaid pitch P2 while yielding a sequential switch from a state in which the first rib 104 is engaged with the first small ribs 106a, 108a to a state in which it corresponds to the rib path 74 and further, to a state in which it is engaged with the second small ribs 106b, 108b.

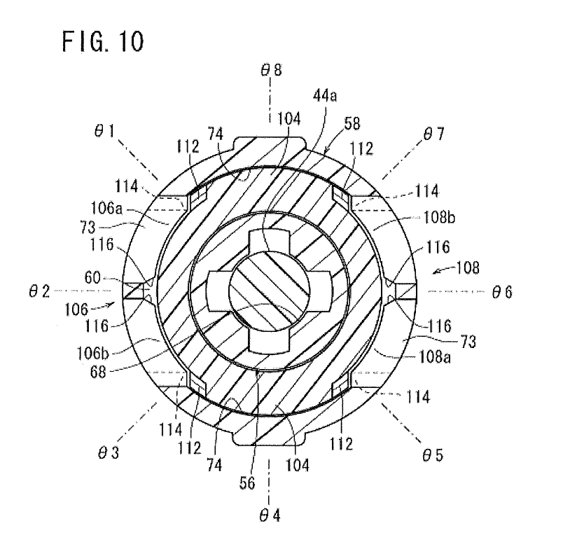

The description below will explain, with reference to FIG. 10 to FIG. 12, in more detail the engagement operation and the movement operation between the pressure portion cylinder 34 and the first pusher 38 by the engagement mechanism 110 constituting the medicine injection device 100.

FIG. 10 shows is a cross-sectional view at a position along the section line X-X in FIG. 9 in a state in which the pressure portion cylinder 34 is inserted in the tubular space 60 of the first pusher 38. In FIGS. 10-12, for convenience of explanation, the tubular space 60 of the first pusher 38, into which the pressure portion cylinder 34 is inserted, is divided into eighths in the circumferential direction and angles from θ1 to θ8 are associated with each. More specifically, in FIGS. 10-12, the second tubular portion 58 is shown fixed in the rotational direction, and for example, the first small rib 106a is arranged between angles from θ1 to θ2, the first small rib 106b is arranged between angles from θ2 to θ3 and the rib path 74 is arranged between angles from θ7 to θ1.

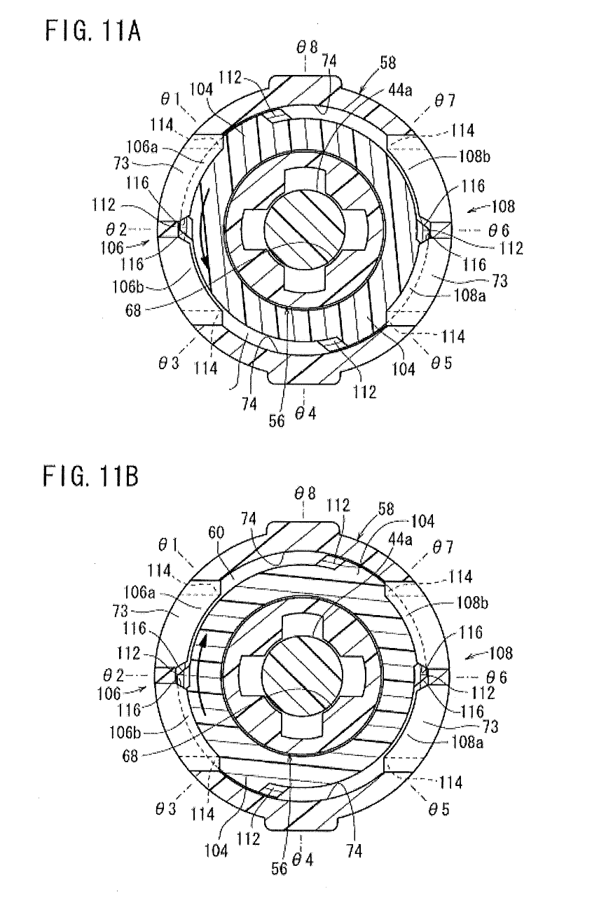

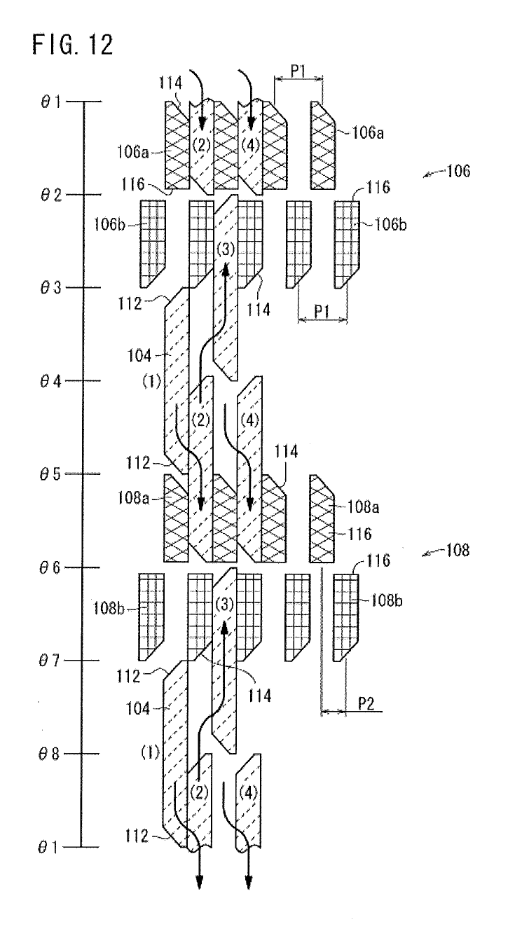

First, the first rib 104 (pressure portion cylinder 34) is rotated counterclockwise as shown in FIG. 11A from a state in which the first rib 104 lies in the position corresponding to the rib path 74 (position (1) of FIG. 12). In that case, the respective inclination surfaces 112, 112 of the respective first ribs 104 move over the inclination surfaces 114, 114 of the second small ribs 106a, 108a of the respective rib sets 106, 108 respectively and thereafter, come into contact with the stopper end portions 116 which lie on the inside of the rib sets 106, 108 of the first small ribs 106b, 108b and which do not have the inclination surface 114, and the further rotation of the pressure portion cylinder is stopped. As a result thereof, the first ribs 104 move from the position (1) to the position (2) in FIG. 12 and stop. Concurrently, the pressure portion cylinder 34 and the first pusher 38 approach each other by an amount as much as the pitch P2 and engage each other, and are fixed in the axial direction. More specifically, the respective first ribs 104 are arranged between angles from θ4 to θ6 and between angles from θ8 to θ2 respectively and the first ribs 104 are engaged with the second small ribs 106a, 108a.

Subsequently, the first ribs 104 (pressure portion cylinder 34) are rotated clockwise as shown in FIG. 11B from a state in which the first ribs 104 lie in the position (2) in FIG. 12 in this manner. In that case, the respective inclination surfaces 112, 112 of the respective first ribs 104 move over the inclination surfaces 114, 114 of the second small ribs 106b, 108b of the respective rib sets 106, 108 respectively and thereafter, come into contact with the stopper end portions 116 of the first small ribs 106a, 108a, and the furthermore rotation of the pressure portion cylinder is stopped. As a result thereof, the first ribs 104 move from the position (2) to the position (3) in FIG. 12 and stop. Concurrently, the pressure portion cylinder 34 and the first pusher 38 approach each other by an amount as much as the pitch P2, engage each other and are fixed in the axial direction. More specifically, the respective first ribs 104 are arranged between angles from θ2 to θ4 and between angles from θ6 to θ8 respectively, and the first ribs 104 engaged the second small ribs 106b, 108b.

Subsequently, in a case in which the first ribs 104 are rotated counterclockwise again from a state in which the first ribs 104 lie in the position (3) in FIG. 12, the first ribs 104 are set to be in the position shown in FIG. 11A (position (4) in FIG. 12) again and are engaged with the first small ribs 106a, 108a. Thereafter, by rotating the pressure portion cylinder 34 and first pusher 38 alternately clockwise and counterclockwise with each other, it is possible to fix the pressure portion cylinder 34 and the first pusher 38 while approaching each other gradually in the axial direction.

This second embodiment of the medicine injection device 100 disclosed by way of example achieves functional effects similar to those associated with the first embodiment of the medicine injection device 10 described above. It is also possible with the second embodiment, by rotating the pressure portion cylinder 34 and the first pusher 38 alternately clockwise and counterclockwise every approximately 180°, to fix the pressure portion cylinder 34 and the first pusher 38 while approaching each other over a distance represented by the pitch P2 which is a half of the pitch P1. Consequently, it is possible to make the hollow gasket 36 progress in the inside of the pressure generating chamber 18 by pulling-in the pressure portion cylinder 34 and the first pusher 38 relatively easily and reliably by a sensory operation depending on only the fingers, without considering the positional relation between the first ribs 104 and the rib sets 106, 108.

The medicine injection device 100 can be set in a state in which the first ribs 104 correspond in position to the rib path 74, and in this state, by pulling-in the pressure portion cylinder 34 and the first pusher 38 toward each other, it is possible to use such a use method by which the medicine is ejected at one time similar to that of a usual syringe.

The invention here is not limited to the disclosed and illustrated examples of embodiments of the medicine injection device. For example, it is possible for the first ribs 48, 104 or the rib sets 70, 72, 106, 108 which are the second ribs to be provided by two pairs or more in the circumferential direction, and also to be employed by only one piece, by three pieces or more without providing one pair in the circumferential direction.

With respect to the first ribs 48, 104 or the rib sets 70, 72, 106, 108, it is possible for each one side thereof to employ only one piece in the axial direction of the tubular portion 44, but in consideration of the engagement strength between the pressure portion cylinder 34 and the first pusher 38, stability on an occasion of the engagement and the like, it is preferable that both are installed by a plurality of pieces.

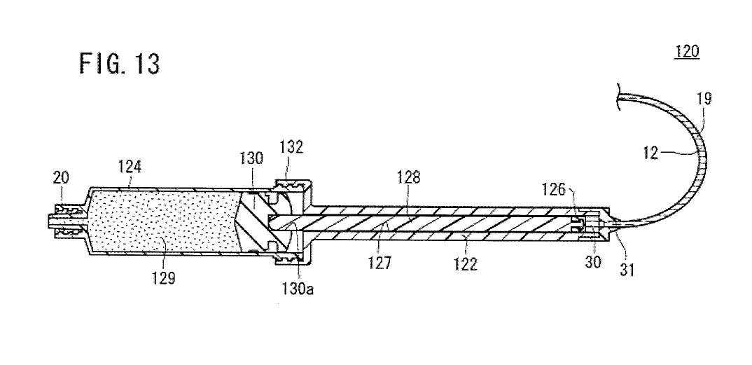

Also, it is possible for the construction of the medicine ejection unit 14 to be other than those shown and described. In brief, it is sufficient if it is a construction capable of appropriately ejecting the medicine from the medicine ejection port by a pressure action from the pressure generation unit 16. By way of example, it is also possible to employ the arrangement shown in FIG. 13.

As shown in FIG. 13, the medicine ejection unit 120 is provided with a first tube body 122 and a second tube body 124 instead of the tube body 24 compared with the medicine ejection unit 14 shown in FIG. 1 and FIG. 2.

With respect to the first tube body (tube body) 122, the flow-in port 31 of the liquid 19 at the distal end thereof is connected with the proximal end of the flexible tube 12 and concurrently, the distal side of a gasket (sub gasket) 126 forming the working chamber 30 is coupled to a rod 128. The rod 128 is inside an action chamber 127, is formed hard by a similar material to that of the second pusher 40, and is coupled to the gasket 126 by insert molding or the like.

With respect to the second tube body (medicine container) 124, the ejection port 20 is provided at the distal end thereof and concurrently, a medicine chamber 129 in which the medicine (bone cement) is accumulated is formed between the ejection port 20 and a cap compatible gasket 130. The proximal end of the second tube body 124 is constituted detachably at a skirt portion (medicine container connection portion) 132 of the distal end of the first tube body 122 and more specifically, regarding the second tube body 124, with the cap compatible gasket 130 functioning as a cap, it functions as a replaceable medicine container (medicine cartridge) with respect to the first tube body 122. The cap compatible gasket 130 includes an attachment hole 130a opened on the proximal side, and when the second tube body 124 is coupled to the first tube body 122, the distal end of the rod 128 is inserted into the attachment hole 130a so as to be coupled to the rod 128.

The medicine ejection unit 120 operates approximately similarly to the medicine ejection unit 14, and when the pressure from the pressure generation unit 16 acts upon the working chamber 30 of the first tube body 122, the rod 128 to which the gasket 126 is coupled progresses or moves forward, the cap compatible gasket 130 connected to the rod 128 moves inside the second tube body 124, whereby it is possible to eject the medicine inside the medicine chamber 129 from the ejection port 20. More specifically, the gasket 126 which functions as the sub gasket relating to the medicine ejection, and the cap compatible gasket 130, are coupled by the rod 128, thereby functioning integrally as a plunger enterable into the second tube body 124 which is connected on the medicine chamber 129 side, and thus it is possible to eject the medicine by the pressure which acts through the liquid 19 from the pressure generation unit 16 side. By using such a medicine ejection unit 120, it is possible to relatively easily replace and use a medicine container of a kind or a volume, which is appropriate for the kind of medicine to be ejected or the volume of a space to be injected and it is possible to further improve versatility and usability of the medicine injection device 10 (100).

The detailed description above describes features and aspects of embodiments of a medicine injection device disclosed as examples of the invention. But the invention is not limited to the precise embodiments and variations described. Changes, modifications and equivalents can be effected by one skilled in the art without departing from the spirit and scope of the invention as defined in the appended claims. It is expressly intended that all such changes, modifications and equivalents which fall within the scope of the claims are embraced by the claims.

What is claimed is:

1. A medicine injection device comprising:
a medicine ejection unit in combination with a pressure generation unit which generates fluid pressure;
the medicine ejection unit comprising: a tubular body possessing an interior partitioned by a sub-gasket slidably arranged in a liquid-tight manner in the interior of the tubular body into a working chamber at a proximal portion of the tubular body and an action chamber distal of the working chamber at a distal end of the tubular body, a fluid flow-in port in fluid communication with the working chamber and connectable to the pressure generation unit to introduce the fluid pressure produced by the pressure generation unit into the working chamber; and a medicine ejection port at a distal end of the tubular body and in fluid communication with the action chamber, the action chamber being configured to receive medicine;
the pressure generation unit comprising: a pressure portion cylinder possessing an interior in which is slidably positioned in a liquid-tight manner a main gasket defining a pressure generating chamber on a distal side of the main gasket; a plunger comprised of first and second coaxial tubular portions spaced apart from one another to define an annular space, the pressure portion cylinder being located in the annular space between the first and second tubular portion so that the first tubular portion is positioned inside the pressure portion cylinder, the first tubular portion possessing a distal end to which the main gasket is fixed so that axial movement of the pressure portion cylinder relative to the first tubular portion causes the pressure portion cylinder to axially move relative to the main gasket; an elongated rod extending inside the first tubular portion and slidably positioned in a liquid-tight manner in a through hole in the main gasket to be moved in a distal direction to generate the fluid pressure which, upon entering the working chamber of the medicine ejection unit, urges the sub-gasket in a distal direction; and
the pressure portion cylinder and the plunger each possessing circumferentially limited ribs which engage one another in a first relative rotational position of the pressure portion cylinder and the plunger to prevent relative axial movement between the pressure portion cylinder and the plunger and which disengage one another in a second relative rotational position of the pressure portion cylinder and the plunger to permit relative axial movement between the pressure portion cylinder and the plunger.

2. The medicine injection device according to claim 1, wherein the second ribs extend inwardly from an inner surface of the plunger, and the first ribs extend outwardly from an outer surface of the pressure portion cylinder.

3. The medicine injection device according to claim 1, wherein the second ribs extend inwardly from an inner surface of the second tubular portion, and the first ribs extend outwardly from an outer surface of the pressure portion cylinder.

4. The medicine injection device according to claim 1, wherein the first tubular portion is a cantilever portion possessing one end portion fixed to the second tubular portion and possessing an opposite end portion that is free.

5. A medicine injection device comprising:
a medicine ejection unit in combination with a pressure generation unit;
the medicine ejection unit comprising: a tubular body possessing an interior partitioned by a sub-gasket slidably arranged in a liquid-tight manner in the interior of the tubular body into a working chamber at a proximal portion of the tubular body and an action chamber positioned distally of the working chamber, and a fluid flow-in port in fluid communication with the working chamber;
the pressure generation unit comprising: a pressure generating chamber which generates fluid pressure communicatable with the working chamber to act upon the working chamber and advance the sub gasket in a distal direction away from the fluid flow-in port; a pressure portion cylinder inside the pressure generating chamber; a first pusher which includes a main gasket slidably disposed in a liquid-tight manner in the pressure portion cylinder and defining an inner volume of the pressure generating chamber, the main gasket possessing a slide hole extending throughout the main gasket; and a second pusher passing through the first pusher and concurrently slidably positioned in a liquid-tight manner in the slide hole of the main gasket to generate the fluid pressure inside the pressure generating chamber by pressing a distal end head portion into the pressure generating chamber; and the pressure portion cylinder and the first pusher are coupled through an engagement mechanism configured to effect fixation between the pressure portion cylinder and the first pusher at a predetermined pitch in an axial direction and to effect fixation release between the pressure portion cylinder and the first pusher permitting relative movement between the pressure portion cylinder and the first pusher in the axial direction.

6. The medicine injection device according to claim 5, further comprising an incompressible fluid which is substantially not compressed by the pressure generated in the pressure generation unit, the incompressible fluid filling the pressure generating chamber.

7. The medicine injection device according to claim 5, wherein:
the pressure portion cylinder communicates with the working chamber at a distal end of the pressure portion cylinder, a proximal portion of the pressure portion cylinder including a tubular member possessing an opening end through which the main gasket is insertable;
the first pusher includes a first tubular portion which possesses a distal end on which the main gasket is fixed and which is insertable into the tubular member; and
the first pusher also includes a second tubular portion forming a tubular space in which the tubular member is positioned so that the tubular member is coaxial with the second tubular portion.

8. The medicine injection device according to claim 7, wherein the engagement mechanism includes:
a first rib on an outer circumferential surface of the tubular member of the pressure portion cylinder and projecting outwardly from a circumferential portion of the outer circumferential surface, the first rib extending circumferentially over a circumferential direction length; and
a plurality of second ribs spaced apart by the predetermined pitch in the axial direction of the second tubular portion on an inner circumferential surface of the second tubular portion of the first pusher, the second ribs projecting inwardly from the inner circumferential surface of the second tubular portion and being individually engageable with the first rib for every pitch of the predetermined pitch in the axial direction of the second tubular portion to effect the fixation between the pressure portion cylinder and the first pusher preventing relative axial movement between the pressure portion cylinder and the first pusher; and
a rib path in the tubular space in which the first rib is positionable to effect the fixation release between the pressure portion cylinder and the first pusher permitting relative axial movement between the pressure portion cylinder and the first pusher; the rib path being circumferentially spaced from the second rib.

9. The medicine injection device according to claim 8, wherein:
the first rib includes a pair of opposing first ribs each of which is a diameter-expanded plate-shaped member possessing an outer diameter greater than a circumferentially adjacent portion of the outer circumferential surface of the tubular member;
each of the second ribs is a diameter-reduced plate-shaped member possessing an inner diameter less than a circumferentially adjacent portion of the inner circumferential surface of the second tubular portion of the first pusher, the second ribs including at least one pair of second ribs facing each other in opposing relation in a diameter direction of the second tubular portion; and
the rib path is positioned between the facing second ribs.

10. The medicine injection device according to claim 8, wherein:
the second ribs are arranged as rib sets each of which includes a first small rib and a second small rib which are circumferentially shorter than the circumferential direction length of the first rib and which are aligned in the circumferential direction of the inner circumferential surface of the second tubular portion;
the first small ribs forming each rib set are arranged to face each other the second small ribs forming each rib set are arranged to face each other; and
in the axial direction of the second tubular portion, the pitch between the first small ribs which are axially adjacent, and the pitch between the second small ribs which are axially adjacent alone are respectively the predetermined pitches with which the first rib is engageable, and the pitches between each first small rib and the axially adjacent second small rib is half the predetermined pitches.

11. The medicine injection device according to claim 10, wherein:
both end portions of the first rib in the circumferential direction of the tubular portion include a first inclination surface whose width narrows toward the proximal side in the axial direction of the tubular portion; and
a second inclination surface at an end portion of the first small rib and the second small rib facing the rib path and constituting one of the rib sets, the second inclination surface possessing a width narrowing toward the distal side in the axial direction of the second tubular portion and configured to permit the first inclination surface to slidably move over the second inclination surface.

12. The medicine injection device according to claim 8, wherein:
the first rib comprises a plurality of first ribs axially spaced apart by the predetermined pitch, and each of the first ribs is engageable with respective ones of the second ribs simultaneously.

13. The medicine injection device according to claim 5, further comprising a medicine ejection port at a distal end of the tube body and in fluid communication with the action chamber of the tubular body.

14. The medicine injection device according to claim 5, further comprising a medicine container connection portion connected to a distal end portion of the tubular body, and wherein the sub gasket includes a plunger enterable into an inside of the medicine container.

15. The medicine injection device according to claim 5, further comprising:
a flexible tube in fluid communication with the working chamber of the medicine ejection unit and the pressure generating chamber of the pressure generation unit; and
wherein an interior of each of the working chamber, the pressure generating chamber and the flexible tube are filled with the incompressible fluid.

16. The medicine injection device according to claim 5, wherein the medicine is bone cement to be injected into the inside of the bone.

17. A method of injecting medicine comprising:
positioning a medicine ejection unit relative to an ejection site, the medicine ejection unit comprising: a tubular body possessing an interior partitioned by a sub-gasket slidably arranged in a liquid-tight manner in the interior of the tubular body into a working chamber at a proximal portion of the tubular body and an action chamber at a distal portion of the tubular body which contains medicine; the working chamber being in fluid communication with a pressure generating chamber of a pressure generation unit, with an incompressible liquid in the working chamber and the pressure generating chamber; the pressure generation unit comprising: a pressure portion cylinder inside the pressure generating chamber; a first pusher which includes a main gasket slidably disposed in a liquid-tight manner in the pressure portion cylinder and defining an inner volume of the pressure generating chamber; and a second pusher passing through the first pusher and concurrently slidably positioned in a liquid-tight manner in a through hole of the main gasket;
relatively rotating the pressure portion cylinder and the first pusher to a fixation position in which the pressure portion cylinder and the first pusher fixed against relative axial movement;
axially moving the second pusher toward the pressure generating chamber while the pressure portion cylinder and the first pusher are in the fixation position to pressurize the incompressible liquid in the pressure generating chamber and cause the pressurized incompressible liquid to act on the sub-gasket through the working chamber to move the sub-gasket in a distal direction and cause ejection of the medicine from the action chamber to the ejection site;
relatively rotating the pressure portion cylinder and the first pusher to a fixation release position in which the pressure portion cylinder and the first pusher fixed are relatively axially movable;
relatively axially moving the pressure portion cylinder and the first pusher;
relatively rotating the pressure portion cylinder and the first pusher to the fixation position to fix the pressure portion cylinder and the first pusher against relative axial movement; and
axially moving the second pusher toward the pressure generating chamber while the pressure portion cylinder and the first pusher are in the fixation position to pressurize the incompressible liquid in the pressure generating chamber and cause the pressurized incompressible liquid to act on the sub-gasket through the working chamber to move the sub-gasket in a distal direction and cause the ejection of additional medicine from the action chamber to the ejection site.
